(19) United States
(12) Patent Application Publication
(10) Pub. No.: US 2018/0305413 A1
(43) Pub. Date: Oct. 25, 2018

(54) Methods for Extracting Phycocyanin

(71) Applicant: Algenol Biotech LLC, Fort Myers, FL (US)

(72) Inventors: Charles Budinoff, Fort Myers, FL (US); Lauren Hehman, Raleigh, NC (US); Kevin Sweeney, Fort Myers, FL (US); Michael D. McConnell, Fort Myers, FL (US); Monica R. Stegman, Fort Myers, FL (US)

(73) Assignee: Algenol Biotech LLC, Fort Myers, FL (US)

(21) Appl. No.: 15/961,591

(22) Filed: Apr. 24, 2018

Related U.S. Application Data
(60) Provisional application No. 62/489,912, filed on Apr. 25, 2017.

(51) Int. Cl. C07K 14/195; C09B 61/00; B01D 11/02; B01D 21/26; B01D 61/14

(52) U.S. Cl. CPC C07K 14/195; C09B 61/00; B01D 2311/2676; B01D 21/262; B01D 61/145; B01D 11/0292

(57) ABSTRACT

Methods for extracting phycocyanin from biomass, comprising suspending dried biomass in a buffer solution, separating the biomass from supernatant, including through centrifugation and/or filtration, and purifying the supernatant, including through filtration.

### CROSS-REFERENCE TO RELATED APPLICATIONS

This application claims the benefit of U.S. Provisional Patent Application No. 62/489,912, filed Apr. 25, 2017, the disclosure of which is incorporated herein by reference in its entirety.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND

Extraction of phycocyanin from fresh biomass that has been dewatered, but not frozen or dried, can involve multiple labor-intensive and costly process steps, including, for example, cell lysis followed by chemical (calcium-phosphate) precipitation or flocculation. Cell lysis by high-pressure homogenization requires a fresh biomass slurry with low to medium viscosity. Higher solid amounts and the presence of non-fragmented cells in fresh biomass increases the total volume of water needed during the process, thus increasing processing time and equipment costs. Chemical precipitation is efficient at removing contaminants and improving product purity, but adds considerable process variability and product loss, requires costly raw materials, and generates significant waste products.

Various approaches to extracting phycocyanin have been the subject of, for example, U.S. Pat. Nos. 9,242,932; 9,131,724; 8,563,701; and 4,320,050; and U.S. Patent Application Publication Nos. 20160130504; 20150239941; and 20140371433.

One currently used method for the isolation of phycocyanin from biomass uses one or more freeze-thaw cycles, which is expensive to employ on a commercial scale. Another common method utilizes various flocculating materials to separate the phycocyanin from the remaining biomass. Kojima et al (U.S. Patent Application Publication No. 2014/0371433) employed the addition of chitosan or one of several commercial flocculants to suspend biomass in order to extract the phycocyanin. However, the addition of these compounds is often costly. Further, this method may result in the presence of at least some of the added compound in the final product. When food-grade phycocyanin is desired, these compounds may not be desirable in the final product. Additionally, when any other products are to be isolated from the waste biomass left over from the phycocyanin preparation, this material may be contaminated with the added compounds and may be difficult to remove, thus reducing their value.

Ben Ouda (U.S. Patent Application Publication No. US2017/0305966) describes the addition of salicylic acid to an aqueous extract of phycocyanin in order to precipitate the phycobiliproteins. However, this results in a substantial amount of salicylic acid in the final product.

An ongoing need exists for an improved method for extracting phycocyanin that minimizes process steps and costs, and increases overall yields.

### SUMMARY

An object of the present invention is a method for extracting phycocyanin from biomass, comprising the steps of suspending dried biomass in a buffer solution to produce a suspension solution, wherein the dried biomass comprises a source organism; separating a biomass residue from an intermediate supernatant; and purifying the intermediate supernatant to produce a product supernatant.

An aspect of the present invention is directed to a method for extracting phycocyanin wherein the microorganism comprises Spirulina.

An additional aspect of the present invention is directed to a method for extracting phycocyanin wherein the suspension solution comprises a biomass concentration (weight per volume) of from about 3% to about 15% in the buffer solution.

An additional aspect of the present invention is directed to a method for extracting phycocyanin comprising the step of stirring the suspension solution at a speed of from about 50 to about 300 rpm for about 4 to about 16 hours in darkness at a temperature of from about 20 to about 50 degrees Celsius before separating the biomass residue from the supernatant.

An additional aspect of the present invention is directed to a method for extracting phycocyanin wherein separating the biomass residue from the supernatant comprises the steps of centrifuging the suspension solution at a first speed to produce a first supernatant, and centrifuging at a second speed and/or filtering the first supernatant to produce the intermediate supernatant.

An additional aspect of the present invention is directed to a method for extracting phycocyanin wherein the suspension solution is centrifuged at a first speed of from about 2,000 to about 20,000 RCF and a temperature of about 20 degrees Celsius for about 2 to about 20 minutes.

An additional aspect of the present invention is directed to a method for extracting phycocyanin wherein the first supernatant is filtered using a microfilter having a pore size of from about 1,000 kDa to about 10 μm.

An additional aspect of the present invention is directed to a method for extracting phycocyanin wherein the first supernatant is filtered using a depth filter having a pore size of from about 0.1 to about 20 μm.

An additional aspect of the present invention is directed to a method for extracting phycocyanin wherein the first supernatant is centrifuged at a second speed of from about 20,000 to about 100,000 RCF and a temperature of about 20 degrees Celsius for from about 10 minutes to about 60 minutes.

An additional aspect of the present invention is directed to a method for extracting phycocyanin wherein purifying the intermediate supernatant comprises filtering the intermediate supernatant using an ultrafilter having a pore size of from about 10 kDa to about 200 kDa.

An additional aspect of the present invention is directed to a method for extracting phycocyanin comprising the step of drying biomass before suspending the biomass in the buffer solution.

An additional aspect of the present invention is directed to a method for extracting phycocyanin wherein the biomass is dried in an oven at a temperature of from about 40 to about 60 degrees Celsius for from about 1 to about 24 hours.

An additional aspect of the present invention is directed to a method for extracting phycocyanin comprising the step of drying the product supernatant to produce a dry phycocyanin product.

An additional aspect of the present invention is directed to a method for extracting phycocyanin wherein purity of the dry phycocyanin product is food grade.

An additional aspect of the present invention is directed to a method for extracting phycocyanin wherein purity, expressed as E1% (“color value”), of the dry phycocyanin product is at least 18.

An additional aspect of the present invention is directed to a method for extracting phycocyanin wherein the recovery of phycocyanin is at least about 60%.

An additional aspect of the present invention is directed to a method for extracting phycocyanin wherein the final phycocyanin product has a purity, measured by the ratio of the absorbance at 620 nm to the absorbance at 280 nm, of at least 1.50.

An additional aspect of the present invention is directed to a method for extracting phycocyanin wherein the residual biomass is substantially free of added chemicals and can thus be utilized to extract additional nutritional components.

An additional aspect of the present invention is directed to a method of extracting phycocyanin wherein the recovery of phycocyanin in the first supernatant is at least 80%.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE SEVERAL VIEWS OF THE DRAWING

Embodiments of the invention will be described below with reference to the following figures.

FIG. 1A and FIG. 1B are two schematic diagrams of embodiments of the phycocyanin purification method. FIG. 1A shows a schematic diagram of a purification method that includes the steps of biomass dehydration, suspension and agitation in the dark, centrifugation, and freeze-drying of the phycocyanin (PC)-rich supernatant. FIG. 1B shows the steps of FIG. 1A plus one or more filtration steps to further purify the phycocyanin.

FIG. 2 shows the absorption spectrum of phycocyanin product, which was carried out in a PerkinElmer Lambda 650 spectrophotometer.

FIG. 3 is a bar graph showing the percentage yield of phycocyanin after suspension in various extraction solutions at several pHs.

FIG. 4 is a bar graph showing the percentage yield of phycocyanin after incubation in suspension solution for various amounts of time (2 hours to 16 hours).

FIG. 5 is an absorbance spectrum of several phycocyanin preparations, as described in Example 8 and shown in Table 1.

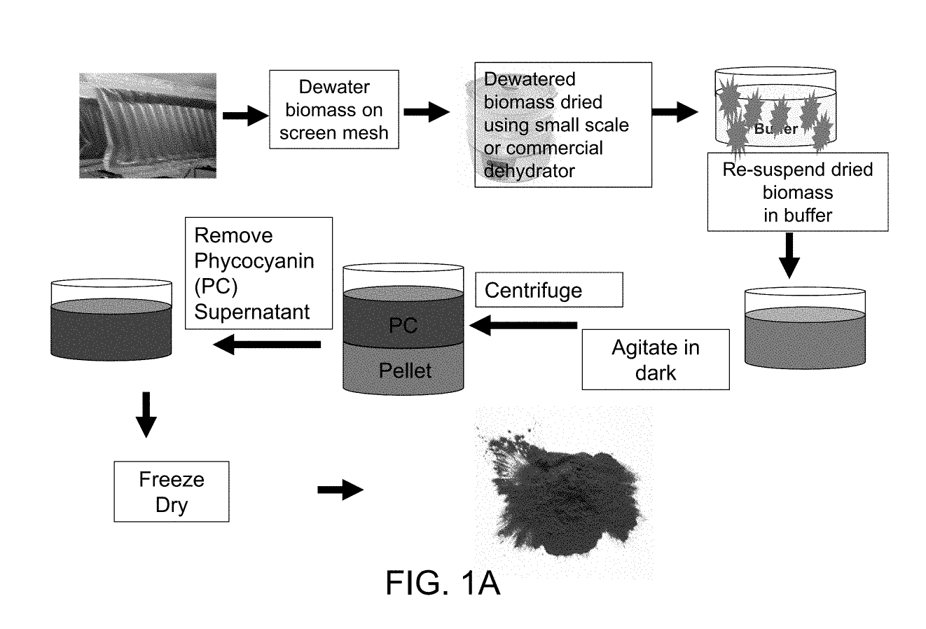

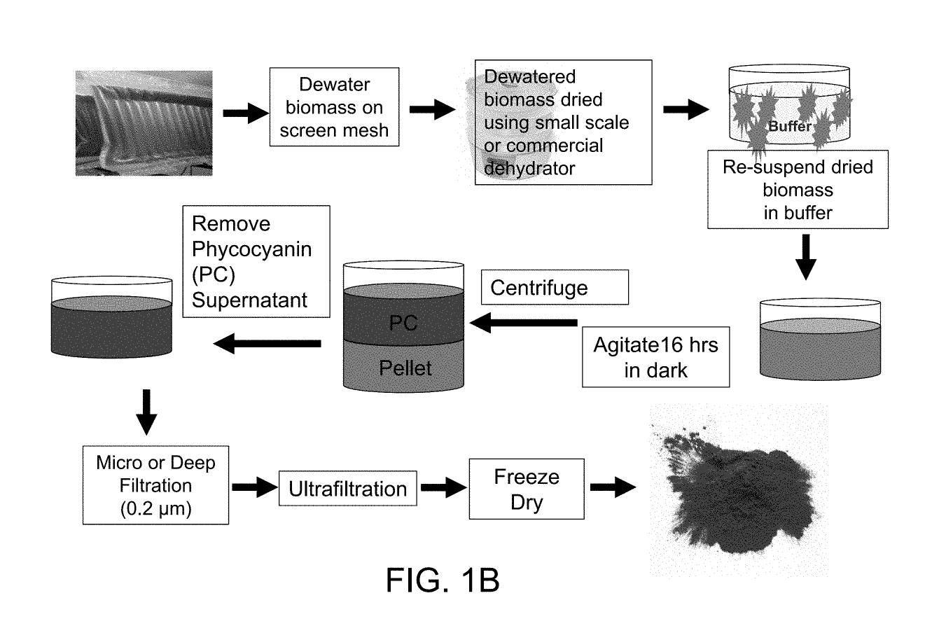

### DETAILED DESCRIPTION

As used herein, the term “source organism” means a microorganism capable of synthesizing phycocyanin. Examples of source organisms are cyanobacteria species present in the genera Spirulina, Arthrospira, Cyanobacterium, and Anabaena. Additional examples of cyanobacterial genera that can be utilized to isolate phycocyanin include but are not limited to Synechocystis, Synechococcus, Acaryochloris, Thermosynechococcus, Chamaesiphon, Chroococcus, Cyanobium, Dactylococcopsis, Gloeobacter, Gloeocapsa, Gloeothece, Microcystis, Prochlorococcus, Prochloron, Chroococcidiopsis, Cyanocystis, Dermocarpella, Myxosarcina, Pleurocapsa, Stanieria, Xenococcus, Arthrospira, Borzia, Crinalium, Geitlerinema, Halospirulina, Leptolyngbya, Limnothrix, Lyngbya, Microcoleus, Cyanodictyon, Aphanocapsa, Oscillatoria, Planktothrix, Prochlorothrix, Pseudanabaena, Starria, Symploca, Trichodesmium, Tychonema, Anabaenopsis, Aphanizomenon, Calothrix, Cyanospira, Cylindrospermopsis, Cylindrospermum, Nodularia, Nostoc, Chlorogloeopsis, Fischerella, Geitleria, Nostochopsis, lyengariella, Stigonema, Rivularia, Scytonema, Tolypothrix, Cyanothece, Phormidium, Adrianema, and the like.

As used herein, the term “biomass” means a mass of organisms, such as, for example, source organisms.

As used herein, the term “phycocyanin” means a pigment-protein complex from the light-harvesting phycobiliprotein family.

As used herein, the term “Spirulina” means a biomass of cyanobacteria comprising Arthrospira platensis and/or Arthrospira maxima.

As used herein, the term “RCF” means relative centrifugal force, or the acceleration in a centrifuge normalized to Earth's gravity.

As used herein, the term “food grade” means of a quality suitable for human consumption, or for use in food production or storage.

As used herein, the terms “yield” and “recovery” mean the phycocyanin present in the final product as a percentage from the total phycocyanin that was present in the initial biomass before dehydration.

As used herein, the term “purity” is the ratio of absorbance at 620 nm to the absorbance at 280 nm. The ratio will be lower when there is more contamination from proteins or other cellular materials that are present in the final product.

As used herein, the term “E1%” or “color value” means purity of a phycocyanin product, which is understood as a percent solution extinction coefficient defined as the absorbance value at 620 nm (1 cm pathlength) of a 1% (weight/volume) solution (abbreviated E1% or E1%/620 The absorbance values are used in equations described by Yoshikawa & Belay, “Single-Laboratory Validation of a Method for the Determination of c-Phycocyanin and Allophycocyanin in Spirulina (Arthrospira) Supplements and Raw Materials by Spectrophotometry”, Journal of AOAC International Vol. 91, No. 3, 2008, to obtain phycocyanin concentrations and purity.

As used herein, the term “about” means approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) above and below the stated value(s) within a confidence interval of 90% or 95%.

The present invention eliminates the need for chemical precipitation, by utilizing a biomass drying step instead of cell lysis. Without wishing to be bound by theory, source organisms are fractured and fragmented during the drying step, and when the dried biomass is suspended in a water-based buffer, phycocyanin leaches into solution while contaminants, which may include, for example, contaminating pigments, proteins, lipids, and other cell constituents, mostly remain within the particulate phase. The dry biomass extract may then be subjected to centrifugation at various speeds and/or filtration using various pore sizes. Utilizing drying instead of cell lysis enables the extraction of higher solids amounts (weight/volume) per batch and increased yields. The method of the present invention achieves a phycocyanin product with food grade purity, defined as E1%≥18.

The biomass can be prepared from a culture of Arthrospira that has been de-watered. The culture is typically de-watered using, for example, a mechanical mesh system, or a commercially available de-watering device. Once de-watered, the wet biomass can be substantially dried using various means, such as those listed below.

In an embodiment, the biomass can be sun-dried, or “solar-dried” in an outdoor environment. Although this may be an inexpensive method of preparing the biomass, particularly at a larger scale, it also may create the highest amount of contamination, as it is likely to take longer to dry than when using the aid of a dehydrator system.

In another embodiment, the biomass can be freeze-dried. Although such systems can be costly, the biomass drying process will likely be faster, and may thus accumulate fewer biological contaminants. Commercially available freeze-dryers or lyophilizers are available for this purpose.

The biomass can also be dried using, for example, a desk top dehydrator designed for food drying. The biomass can also be dried using a commercial scale dehydrator, or a spray-drying apparatus. Examples of suitable types of dehydration systems include, for example, spray dryers, cabinet dryers, tunnel dryers, refractive window dryers, and continuous flow belt dryers. Examples of commercially available small and large scale dehydrators include, for example, the Weston 28-1001-W 10-Tray Food Dehydrator, The Santiam Tray/Tunnel Dryer, the Columbia Tunnel Dryer, and the Excalibur Ss Commercial Food Dehydrator 2 Zone Nsf, Model# COM2.

The biomass can be dried at a temperature range of from about 15° C., 20° C., 25° C., 30° C., 35° C., 40° C., 55° C., to about 60° C. In an embodiment, the drying temperature is about room temperature. In another embodiment, the drying temperature is about 55° C.

The biomass can be dried for a range of time from about 1, 2, 4, 6, 8, 12, 18, or 24 hours, or more. The biomass can be dried to a moisture content of from about 1% to about 10%. In an embodiment of the invention, the moisture content of the dried biomass is about 4% to about 7%. The biomass, once dried, can be stored for a time prior to the suspension step.

Various buffers can be used to suspend the dried biomass. The buffer concentration can be from about 10 mM, 50 mM, 100 mM, 200 mM, 300 mM, 400 mM, to about 500 mM. If the final phycocyanin is to be “food-grade”, then the buffer can be chosen to be acceptable for food-grade preparations. In an embodiment, the buffer is chosen from citrate, citrate disodium phosphate buffer, sodium phosphate buffer, and potassium phosphate buffer. Additionally, a number of commercially available, synthetic buffer systems are available, such as MES, MOPSO, MOPS, BES, TES, HEPES, and others, although many of these may not be suitable for food-grade preparations.

The pH of the suspension buffer can also be adjusted as desired. A pH of from about 5.0, 5.5, 6.0, 6.5, 7.0, 7.5, or 8.0 can be used. In an embodiment, a pH of about 6.0 is used for the suspension buffer.

Various agitation schemes and temperatures can be used to mix the suspended biomass in order to extract more phycocyanin in a shorter period. In an embodiment, the suspension mixture is stirred at 125 rpm at 30° C. The suspension mixture can also be mixed by stirring, for example, at a range of about 50 rpm, 100 rpm, 200 rpm, 300 rpm, 400 rpm, to about 500 rpm or more, at a temperature range from about 20° C., 25° C., 30° C., 35° C., to about 40° C.

The suspension period optimally occurs in darkness or in very low light, in order to decrease the light-related degradation of the phycocyanin. The suspension period can be, for example, about 1, 2, 3, 5, 8, 10, 12, 14, 16, 18, 21, or 24 hours or more. Much of the phycocyanin leaches into the buffer by about 8 hours. Based on the particular buffers, pH, and soak times, almost all of the phycocyanin that can be removed from the biomass will be in the buffer solution. The longer the process runs, the greater the possibility of contamination by heterotrophic organisms which can lessen any benefit of extracting any remaining phycocyanin from the mixture.

The phycocyanin that has leached out of the cellular material can then be separated by various means from the remaining cellular material. This separation can involve, for example, steps of centrifugation and filtration.

In an embodiment, the material is separated using a centrifuge. The centrifugation can vary by temperature, time, and speed. These factors can be varied based on the desired purity of the final product, as well as the type of centrifugation system available. The material can be centrifuged, for example from about 5 minutes to about 120 minutes, at a speed of from about 1,000 RCF to about 20,000 RCF to produce a first supernatant. The temperature can be, for example, from about 4° C., 10° C., 15° C., 20° C., to about 25° C. In an embodiment, the suspension is centrifuged at 10,000 RCF for about 10 minutes at 20° C.

In an embodiment, the material is treated to a filtration step. In an embodiment, the filtration step is microfiltration or depth filtration. The filtration step can be, for example, a microfiltration unit having a pore size range of from about 0.2 to about 2.0 μm. In an embodiment, the first supernatant produced as described in the above paragraph is treated to a filtration step using the 0.2 μm pore size. In another embodiment, the first supernatant is treated to a stepwise filtration process, such as from 2.0 μm, then 1.0 μm, then 0.45 μm, then 0.2 μm. This results in an “intermediate supernatant”.

An ultrafiltration unit can also be employed to further purify the phycocyanin-rich “intermediate supernatant”. As an example, the “intermediate supernatant produced in the above paragraph can be treated to the ultrafiltration unit. Typically, the ultrafiltration unit has a pore size of from about 10 kDa to about 100 kDa. In an embodiment, the pore size is about 100 kDa. This produces a phycocyanin-rich “product supernatant” which can then be dried. The drying can be performed by use of commercially available methods to include freeze-drying, spray drying, refractive window drying, and other known drying technologies.

In another embodiment of the invention, in addition to the phycocyanin extraction, the remaining residual biomass can also be utilized for isolation of value-based components, such as nutritionally useful compounds, cosmetics, and pharmaceuticals. This is because no flocculants or other added chemicals are used, so that all of the material is food grade. Example 9 and Table 2 describe the potential nutrients present in the residual biomass. Importantly, other methods of purification of phycocyanin require the use of a flocculant, which is likely to contaminate the residual biomass so that it cannot be used, or so that it requires additional steps for isolation of the component. Utilization of not only the phycocyanin pigment, but also other valuable components in the biomass can be both environmentally friendly and cost effective.

### EXAMPLES

### Example 1

### Preparation of Dried Biomass

Wet Arthrospira biomass (85-90% moisture content) was dried in a vertical flow dehydrator with an air temperature of about 48 degrees Celsius for about 24 hours.

### Example 2

### Suspension of Dried Arthrospira Biomass in Citrate Buffer

A first suspension solution having a biomass concentration of 3.2% (weight per volume) was prepared from 25.8 grams of dried (dehydrated) Arthrospira biomass in 800 mL of 50 mM citrate buffer, pH=6. A second suspension solution having a biomass concentration of 6.4% (weight per volume) was prepared from 51.6 grams of dried (dehydrated) Arthrospira biomass in 800 mL of 50 mM citrate buffer, pH=6. Other buffer solutions suitable for use with the present invention will have a pH of from about 5 to about 7.5, and include, for example, citric acid buffers, phosphate buffers and Good's buffer solutions.

Each suspension solution was incubated in covered, dark conditions for 16 hours at 30 degrees Celsius while being stirred at 125 revolutions per minute, or rpm. After incubation, each suspension solution was centrifuged for 10 minutes at 10,000 RCF and 20 degrees Celsius to produce a product supernatant. Phycocyanin yield from each product supernatant was measured.

Total phycocyanin recovery from the first suspension solution was 1,964.5 mg. Total phycocyanin recovery from the second suspension solution was 4,233.0 mg.

### Example 3

### Preparation of Phycocyanin by Mixing a Suspension of Dried Arthrospira Biomass in Citrate Buffer Followed by Centrifugation, Filtration, and Freeze-Drying

A suspension solution having a biomass concentration of about 3.2% (weight per volume) was prepared from 32.3 grams of dried (dehydrated) Arthrospira biomass in 1,000 mL of 50 mM citrate buffer, pH=6. The suspension solution was incubated in covered, dark conditions for 16 hours at 30 degrees Celsius while being stirred at 125 rpm.

After incubation, the suspension solution was centrifuged for 10 minutes at 10,000 RCF and 20 degrees Celsius using a Beckman Coulter Avanti JXN-26 centrifuge equipped with a JLA-8.1000 roter, to produce a first supernatant. The first supernatant was filtered through a microfiltration unit having a pore size of 0.2 μm to produce an intermediate supernatant. The intermediate supernatant was filtered through an ultrafiltration unit having a pore size of 100 kDa to produce a product supernatant. The product supernatant was dried in a freeze dryer to produce the dry product.

Phycocyanin yield from the first supernatant was measured at 99%. Phycocyanin yield of the dried product was measured at 67%. Phycocyanin purity (E1%) in the dried product was 19.05.

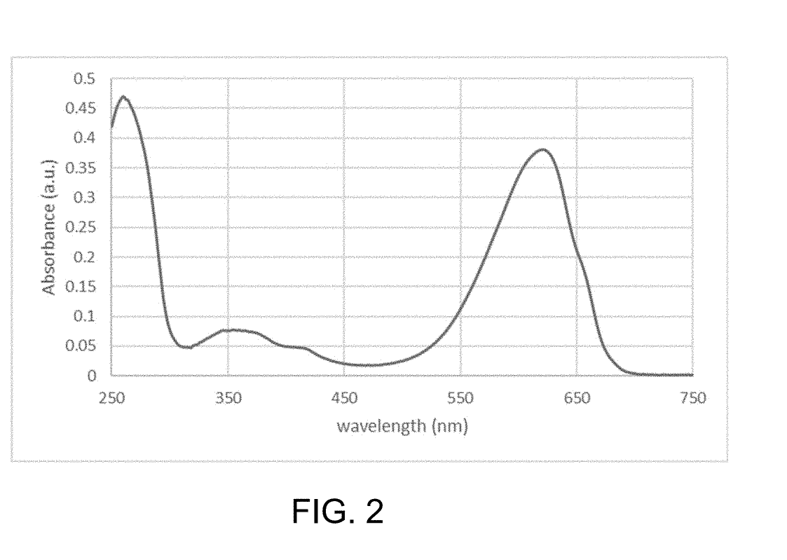

Phycocyanin concentration and purity was determined by measuring the absorbance of the product supernatant diluted in buffer or of the dry product dissolved in buffer at 650 nm and 620 nm using a spectrophotometer. A plot of the absorbance scan is shown in FIG. 2.

### Example 4

### Stability of Phycocyanin in Supernatant of Replicate Samples

Five replicates of a suspension solution having a biomass concentration of about 3.2% (weight per volume) were prepared from 26.33 grams of dried (dehydrated) Arthrospira biomass in 800 mL of 50 mM citrate buffer, pH=6. Each suspension solution was incubated in covered, dark conditions for 16 hours at 30 degrees Celsius while being stirred at 125 rpm. After incubation, each suspension solution was centrifuged for 10 minutes at 10,000 RCF and 20 degrees Celsius to produce a product supernatant. Mean phycocyanin yield in the product supernatants was approximately 88%. The results show that the process can be replicated successfully with similar yields.

### Example 5

### Preparation of Phycocyanin: Effect of Modified Centrifugation Parameters

The following experiment was performed to determine if longer centrifugation at a higher speed could replace filtration through a 0.2 μm pore size filter described in Example 3. A suspension solution containing about 3.2% (weight per volume) of dried (dehydrated) Arthrospira biomass in 50 mM citrate buffer, pH=6, was stirred at 125 rpm in the dark for 16 hours at 30° C. The material was then centrifuged for 90 minutes at 80,000 RCF and 20 degrees Celsius to produce an intermediate supernatant. The intermediate supernatant was filtered through an ultrafiltration unit having a pore size of 50 kDa to produce a product supernatant. The product supernatant was dried in a freeze dryer to produce dry phycocyanin product. Phycocyanin purity (E1%) in the dry phycocyanin product was 22.6.

### Example 6

### Effect of Other Extraction Solutions at Various pH Ranges

In addition to citrate buffer, phycocyanin was extracted from dried biomass using other suspension buffers. The dehydration-dried biomass from Example 1 was suspended in a 3.2% weight/volume amount with the buffers below:
a. 50 mM citrate buffer (pH 5.0, 5.5, or 6.0),
b. 50 mM citrate disodium phosphate buffer (CDP) (pH 5.0, 5.5, 6.0, 6.5, or 7.0), and
c. 50 mM potassium phosphate buffer (pH 6.0, 6.5, 7.0, or 7.5).

The samples were stirred at 125 rpm for 16 hours at 30° C. in the dark. Samples were then centrifuged at 7,000 rpm for 10 minutes at 20° C. The supernatant was taken for further measurements. Purity of the extracted phycocyanin was measured by the spectrophotometric absorbance ratio of 620 nm:280 nm. The percentage yield of phycocyanin was also determined by comparing the amount of phycocyanin present in the supernatant to the amount of phycocyanin in the pelleted biomass.

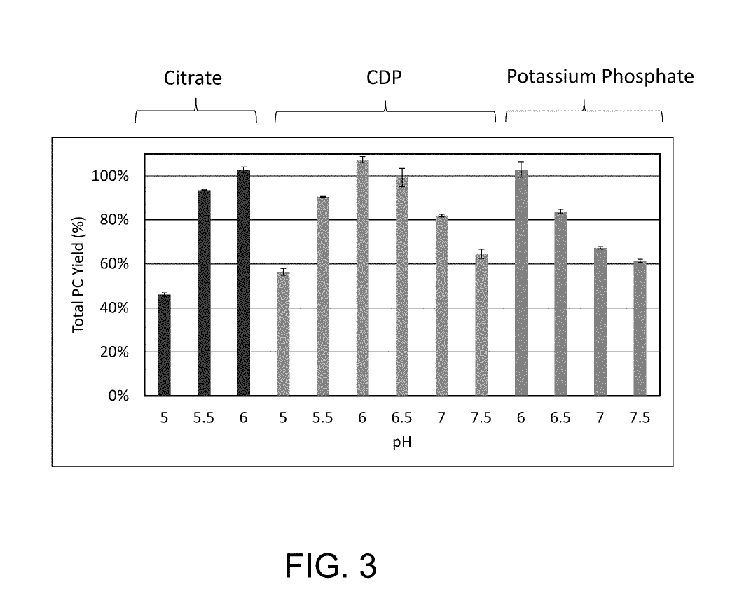

As shown in FIG. 3, pH 6.0 resulted in the highest phycocyanin yield, regardless of the buffer used. At pH 6.0 all three buffers appeared to have a near 100% extraction yield.

### Example 7

### Determination of Optimal Extraction Time

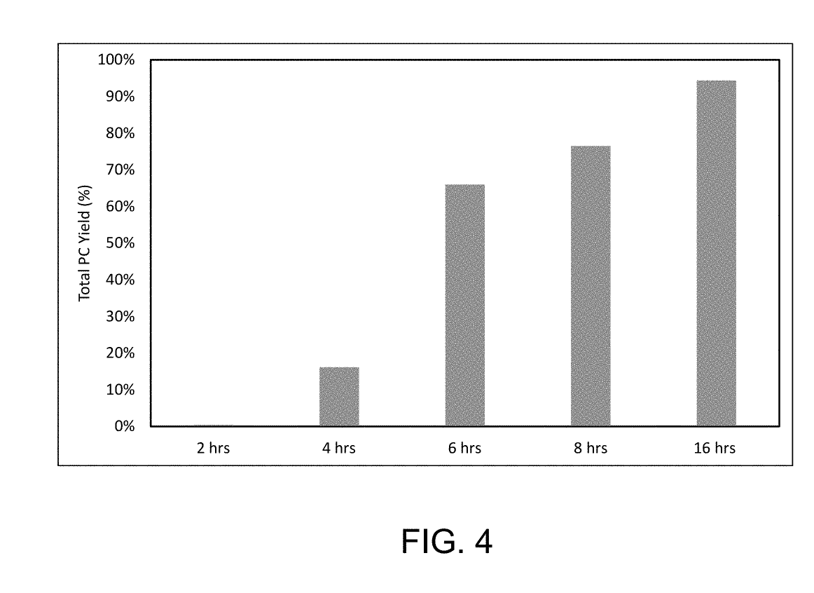

To determine the optimal amount of time needed for extraction of phycocyanin, a dehydration-dried sample of Arthrospira (prepared as described in Example 1) was suspended in a 3.2% weight/volume amount of 50 mM Potassium Phosphate, pH 6.0, and stirred at 125 rpm for 16 hours at 30° C. in the dark. Samples of the suspended material were assayed at 2, 4, 6, 8, or 16 hours. The percentage of phycocyanin yield (in comparison to that of dried biomass) was determined (FIG. 4). It was determined that the 16 hour extraction time produced the most phycocyanin at greater than 90%. However, the 8 hour time period resulted in a high level of extraction (˜75%) while lessening the likelihood of increased contamination of the suspension.

### Example 8

### Purity and E1% Value of Phycocyanin Prepared by Various Methods

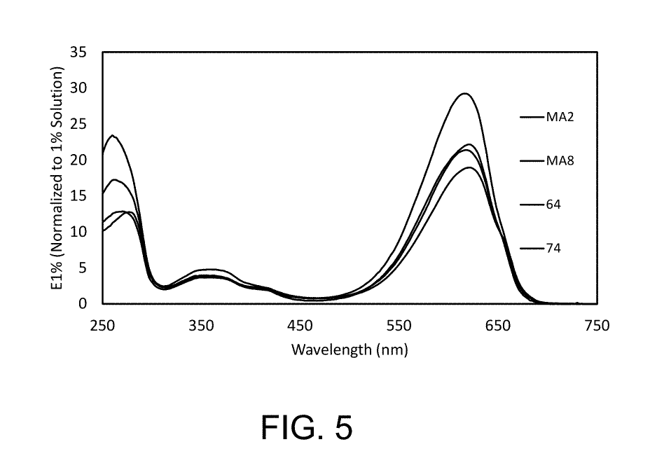

Food-grade phycocyanin needs to have a certain amount of purity and E1%. Several of the above phycocyanin extraction and purification methods were compared for the final E1%. Table 1 (below) lists the purity information for the various samples. FIG. 5 shows the absorbance spectrum of several of the samples.

TABLE 1

| Sample ID and | Total PC Recovered | | Purity |
|---|---|---|---|
| Parameters | (%)* | E1% | (620:280) |
| TB2: CDP | 61.8 | 21.5 | 1.49 |
| buffer | | | |
| MA2: CDP | 60.8 | 19.3 | 1.77 |
| buffer | | | |
| MB2: CDP | 73.2 | 26.6 | 1.86 |
| buffer | | | |
| MA8: CDP | 62.2 | 30.7 | 2.34 |
| buffer | | | |
| MB8: CDP | 56.8 | 29.9 | 2.26 |
| buffer | | | |
| Batch 64: | 84.5 | 19.0 | 1.06 |
| Citrate buffer | | | |
| Batch 74: CDP | 68.0 | 20.7 | 1.39 |
| buffer | | | |

### Example 9

### Nutritional Value of Solids from Centrifugation Step

The above-mentioned centrifugation steps not only result in purified phycocyanin, but also results in a nutrition-dense solid material that can be used for many food products. The solid precipitate from the centrifugation step was analyzed for nutritional content using standard methods. In contrast to phycocyanin purification methods that utilize various chemical flocculants, the precipitate from this method is a fully recoverable nutrition-dense product with no contaminating chemicals.

TABLE 2
Nutritional value of biomass precipitate after centrifugation

| | Per Serving | | |
|---|---|---|---|
| Analysis | (100 g) | | Analysis Method |
| Protein | 66.1 | g | AOAC 2001.11 |
| Total Carbs | 9.27 | g | SAM 07006 (Calculation) |
| Total Fat | 6.88 | g | SAM 05001 (Gravimetric) |
| Ash | 13.05 | g | SAM 07034 (Gravimetric) |
| Moisture | 4.7% | | USP 40 <921> Met. III |
| Calories | 363 | calories | AOAC |
| Fatty Acid Analysis: | | | SAM 05003 (GC-FID) |
| Palmitic Acid | 4075 | mg | SAM 05003 (GC-FID) |
| Palmitoleic Acid | 503 | mg | SAM 05003 (GC-FID) |
| Stearic Acid | 62.3 | mg | SAM 05003 (GC-FID) |
| Oleic Acid | 258 | mg | SAM 05003 (GC-FID) |
| Linoleic Acid | 2264 | mg | SAM 05003 (GC-FID) |
| gamma-Linolenic | 2771 | mg | SAM 05003 (GC-FID) |
| Acid | | | |

Example embodiments have been described herein for illustrative purposes only and are not limiting. Other embodiments are possible and are covered by the disclosure and the teachings contained herein. The breadth and scope of the disclosure should not be limited by any of the above-described embodiments, but should be defined only in accordance with features and claims supported by the present disclosure and their equivalents. Moreover, embodiments of the subject disclosure may include formulations, compounds, methods, systems, and devices which may further include any and all elements/features from any other disclosed formulations, compounds, methods, systems, and devices, including the manufacture and use thereof. Features from one and/or another disclosed embodiment may be interchangeable with features from other disclosed embodiments, which, in turn, correspond to yet other embodiments. One or more features/elements of disclosed embodiments may be removed and still result in patentable subject matter (and thus, resulting in yet more embodiments of the subject disclosure). Furthermore, some embodiments of the present disclosure may be distinguishable from the prior art by specifically lacking one and/or another feature, functionality, ingredient or structure, which is included in the prior art (i.e., claims directed to such embodiments may include “negative limitations” or “negative provisos”).

All references, articles, publications, patents, patent publications, and patent applications cited herein are incorporated by reference in their entireties for all purposes. Mention of any reference, article, publication, patent, patent publication, and patent application cited herein is not an acknowledgment that they constitute valid prior art or form part of the common general knowledge in any country in the world.

What is claimed is:

1. A method for extracting phycocyanin from biomass, comprising the steps of:
a) suspending dried biomass in a buffer solution to produce a suspension solution, wherein the dried biomass comprises a source organism;
b) separating a biomass residue from an intermediate supernatant; and
c) purifying the intermediate supernatant to produce a product supernatant.

2. The method of claim 1, wherein the microorganism comprises Spirulina.

3. The method of claim 1, wherein the suspension solution comprises a biomass concentration (weight per volume) of from about 3% to about 15% in the buffer solution.

4. The method of claim 1, further comprising the step of stirring the suspension solution at a speed of from about 50 to about 300 rpm for about 4 to about 16 hours in darkness at a temperature of from about 20 to about 50 degrees Celsius before separating the biomass residue from the supernatant.

5. The method of claim 1, wherein separating the biomass residue from the supernatant comprises the steps of centrifuging the suspension solution at a first speed to produce a first supernatant, and centrifuging at a second speed and/or filtering the first supernatant to produce the intermediate supernatant.

6. The method of claim 5, wherein the suspension solution is centrifuged at a first speed of from about 2,000 to about 20,000 RCF and a temperature of about 20 degrees Celsius for about 2 to about 20 minutes.

7. The method of claim 5, wherein the first supernatant is filtered using a microfilter having a pore size of from about 1,000 kDa to about 10 μm.

8. The method of claim 5, wherein the first supernatant is filtered using a depth filter having a pore size of from about 0.1 to about 20 μm.

9. The method of claim 5, wherein the first supernatant is centrifuged at a second speed of from about 20,000 to about 100,000 RCF and a temperature of about 20 degrees Celsius for from about 10 minutes to about 60 minutes.

10. The method of claim 1, wherein purifying the intermediate supernatant comprises filtering the intermediate supernatant using an ultrafilter having a pore size of from about 10 kDa to about 200 kDa.

11. The method of claim 1, further comprising the step of drying biomass before suspending the biomass in the buffer solution.

12. The method of claim 11, wherein the biomass is dried in an oven at a temperature of from about 40 to about 60 degrees Celsius for from about 1 to about 24 hours.

13. The method of claim 1, further comprising the step of drying the product supernatant to produce a dry phycocyanin product.

14. The method of claim 13, wherein purity of the dry phycocyanin product is food grade.

15. The method of claim 13, wherein purity, expressed as E1% (“color value”), of the dry phycocyanin product is at least 18.

16. The method of claim 15, wherein the recovery of phycocyanin is at least about 60%.

17. The method of claim 15, wherein the final phycocyanin product having a purity, measured by the ratio of the absorbance at 620 nm to the absorbance at 280 nm, of at least 1.50.

18. The method of claim 1, wherein the biomass residue is substantially free of contaminating compounds.

19. The method of claim 1, wherein the recovery of phycocyanin in the first supernatant is at least 80%.
